(19) United States
(12) Patent Application Publication
(10) Pub. No.: US 2002/0041854 A1
(43) Pub. Date: Apr. 11, 2002

(54) Cosmetic compositions in powder form comprising a binder, and make-up and cosmetic care methods

(76) Inventors: Anke Hadasch, Paris (FR); Patricia Lemann, Creteil (FR); Jean-Thierry Simonnet, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/860,567

(22) Filed: May 21, 2001

(30) Foreign Application Priority Data
May 19, 2000 (FR) 00 06448

(51) Int. Cl. A61K 7/021

(52) U.S. Cl. 424/63

(57) ABSTRACT

A cosmetic composition, such as a make-up and/or cosmetic care composition, in powder form comprising a pulverulent phase and a binder in said powder composition, wherein the binder is chosen from compositions comprising an aqueous continuous phase, and the cosmetic applications, in make-up and/or in cosmetic care, of such a composition.

The present invention relates to make-up and/or cosmetic care compositions in powder form comprising a binder. These make-up and/or cosmetic care compositions include, but are not limited to, compact, pressed or cast powders.

Make-up powders may comprise a pulverulent phase comprising pigments and fillers and a dispersing, i.e. binder, phase comprising fatty substances. The dispersing (binder) phase, which may also be called a fatty phase, may impart at least one of the following attributes to the make-up products: homogeneous dispersion of the pulverulent compounds, facilitation of pulverulent compounds' cohesion in the final composition, adjustment of the density of the finished product, provision of softness and emollient properties to the cosmetic product, and promotion of the cosmetic product's adhesion to the skin.

The development of dispersing, i.e. binder, phases in make-up products may raise many difficulties.

For example, in the case of powders, the final composition should be sufficiently uniform and compact so as to have a good capacity to be taken up and to avoid fragmentation caused, for example, by impacts.

It is possible to use a mixture of mineral and plant oils combined with esters as a binder phase in powders. However, the products comprising these esters may lead to products lacking softness or poor dispersion of the pulverulent phase.

It is also possible to use silicone oils, which may provide at least one property chosen from slipperiness, softness and spreading ease, but staying power and impact resistance of the make-up may be adversely affected.

Additionally, certain perfluoro oils, for example, perfluoropolyethers, are known to provide softness to cosmetic compositions, such as powders. However, the product formulator is then often confronted with problems of insolubility of these oils in the usual hydrocarbon-based oils or silicone oils. This insolubility may lead to problems of instability of the composition. Moreover, compositions comprising perfluoro oils may have mediocre impact strength properties. Some of these oils may also have the drawback of poor dispersion of the pulverulent phase and can lead to relatively low color intensity for the pigments, possibly resulting in a “white effect”, which is not desirable, aesthetically speaking.

Make-up powders are thus products generally comprising a high content of pulverulent compounds. These compounds may be qualified as being of the “dry” or “oil” type. These products may impart sensations of tautness or dryness when they are applied to the skin. However, due to the high content of pulverulent compounds, it may be difficult to introduce large amounts of water into such products. The reason for this is that the water does not bind the particles, but rather, the water aggregates with the pulverulent compounds to form an unstable heterogeneous paste or mix. This paste is unsuitable to use as a make-up product since a measured amount of product cannot be taken up. Moreover, a powdered product made from a sticky paste or mix, may result in a plaster-like complexion, rather than the desired velvety appearance.

Another drawback to introducing water into make-up powders is that it is difficult to incorporate active agents, such as, hydrophilic active agents, into the powders and still maintain a stable composition.

Now, women increasingly wish to use make-up products which not only enhance their beauty but also give the skin a fresh sensation or a care effect.

Thus, it would be particularly useful to be able to introduce an aqueous phase into make-up powders.

The inventors have found, unexpectedly, that the use of a composition comprising an aqueous continuous phase as a powder binder makes it possible to obtain a powder which not only can give a sensation of freshness and moisturization, but also can have effective cosmetic care and/or make-up properties.

One embodiment of the invention is thus a make-up and/or cosmetic care composition, in the form of a powder, comprising a pulverulent phase and a binder, wherein the binder is chosen from compositions comprising an aqueous continuous phase.

The compositions according to the invention can have the advantage of being able to comprise, for example, large amounts of water or hydrophilic active agents while at the same time remaining powders that are easy to disintegrate and are comfortable to apply. Despite the presence of water or hydrophilic active agents, no formation of a heterogeneous paste with a plaster-like effect may be observed. A cosmetic powder which has good cohesion and which may be easily used for applying make-up is obtained.

The compositions thus obtained can bring about a pleasant sensation of freshness when applied. They may moisturize the skin and not give a sensation of tautness or dryness and they may effectively disperse the pigments. The composition can be homogeneous and may remain so even several hours after it has been applied to the skin.

In powder form, the compositions of the present invention can exhibit effective staying power by minimizing the transfer and migration of the powder into the folds of the skin.

The compositions according to the invention may also have at least one of the following cosmetic properties: adequate skin adherence (adhere to the skin enough but not too much), softness and easy application.

The present invention also relates to a cosmetic process for treating (making up, or caring for) human keratinous materials such as skin and body, comprising application of the compositions as defined above to these materials.

According to the present invention, the term “keratinous materials” refers to skin, nails, hair, eyelashes, eyebrows, mucous membranes (inner edge of the lower eyelids), semi-mucous membranes (lips), and any other area of body and facial skin.

Another aspect of the invention is the use of at least one composition comprising an aqueous continuous phase, in a make-up and/or cosmetic care composition in powder form, to improve the moisturization imparted by such composition on keratin materials. Such a composition can improve the moisturization of such keratin materials.

Another aspect of the invention is the use of at least one composition comprising an aqueous continuous phase, in a make-up and/or cosmetic care composition in powder form, to improve the development of color in the composition. Such a composition can improve the development of color.

It is possible, by virtue of the present invention, to prepare compositions comprising a smaller quantity of pigment than is typically found in conventional compositions, while at the same time attaining the same intensity of color. Similarly, by using the same quantity of pigments as is typically found in conventional compositions, use of the binder according to the invention makes it possible to obtain a composition whose color is much brighter, more intense and more developed.

The compositions according to the invention may be advantageously used in the fields of make-up for and care of human keratinous materials, such as, for example, skin and mucous membranes. Non-limiting examples of make-up products for the face and the skin are chosen from eye shadows, blushers, face powders, body powders, concealer products, foundations, make-up products for the body, lipsticks, mascaras, eyeliners, anti-sun products and self-tanning products.

Other characteristics, aspects and advantages which may pertain to at least one embodiment of the present invention will become apparent on reading the detailed description which follows.

The compositions according to the invention comprise a pulverulent phase which may comprise at least one pulverulent chosen from pigments, nacres, fillers, and flakes usually used in cosmetic compositions.

The term “pigment” may be understood to indicate particles intended to color and optionally opacify the composition of the present invention. These pigments may be white or colored and made from mineral or organic origin. The pigments are insoluble in the medium of the compositions according to the invention.

The pigments may be present in an amount, for example, ranging from 0.05% to 80% by weight relative to the total weight of the composition, such as from 0.5% to 50%. The pigments may be white or colored and from mineral or organic origin. The pigments may also be of usual size or of nanometric size.

Non-limiting representatives of mineral pigments and nanopigments may be chosen from titanium dioxide, zirconium dioxide, cerium dioxide, zinc oxide, iron oxide, chromium oxide, nanotitaniums, nanozincs and ferric blue. Non-limiting representatives of organic pigments may be chosen from carbon black and lakes, such as, for example, calcium salts, barium salts, aluminium salts and zirconium salts, and acidic dyes, such as, for example, halo acid dyes, azo dyes and anthraquinone dyes.

The pigments may be coated with at least one compound chosen from silicone compounds such as, for example, polydimethylsiloxanes and polymers, such as polyethylenes. Mention may thus be made of the “SI oxides” which are polymethyl-hydrogenosiloxane-coated pigments sold by the company MIYOSHI.

For the purposes of this invention, the term “filler” is understood to indicate colorless and white, lamellar and non-lamellar, particles of mineral or synthetic origin which may provide at least one of the following properties to the composition: body, rigidity, make-up softness, make-up matt effect and make-up uniformity.

The fillers, which may be present in the composition in an amount, for example, ranging from 0.05% to 99%, such as from 0.1% to 99%, further such as 70% to 99.9%, and even further such as 77% to 98%, by weight relative to the total weight of the composition may be lamellar and non-lamellar particles of mineral or synthetic origin. Non-limiting examples of fillers may be chosen from talc, mica, silica, kaolin, Nylon powder, poly-β-alanine powder, polyethylene powder, Teflon, lauroyllysine, starch, boron nitride, bismuth oxychloride, tetrafluoroethylene polymer powders, polymethyl methacrylate powders, polyurethane powders, polystyrene powders, polyester powders, synthetic hollow microspheres, such as, for example, Expancel (Nobel Industrie), microsponges, such as Polytrap (Dow Corning), silicone resin microbeads (for example Tospearls from the company Toshiba), zinc oxide, titanium oxide, zirconium oxide, cerium oxide, precipitated calcium carbonate, magnesium carbonate, magnesium hydrocarbonate, hydroxyapatite, hollow silica microspheres (silica beads from the company Maprecos), glass microcapsules, ceramic microcapsules, metal soaps derived from organic carboxylic acids comprising from 8 to 22 carbon atoms, such as from 12 to 18 carbon atoms, for example, zinc stearate, magnesium stearate, lithium stearate, zinc laurate and magnesium myristate.

The term “nacre” may be understood as indicating iridescent particles which reflect light.

The nacres may be present in the composition according to the invention, in an amount, for example, ranging from 0.05% to 80% by weight relative to the total weight of the composition, for example in an amount ranging from 2% to 50% by weight. Non-limiting representatives of the nacres which may be used in the composition according to the invention, may be chosen from natural mother-of-pearl, mica coated with titanium oxide, mica coated with iron oxide, mica coated with natural pigment, mica coated with bismuth oxychloride, and colored titanium mica.

The compositions according to the invention may further comprise flakes.

In the compositions according to the invention, the pulverulent phase may be present in an amount, for example, of at least 50% by weight relative to the total weight of the composition. Alternatively, the pulverulent phase may be present in an amount of at least 70% by weight relative to the total weight of the composition. Further, the pulverulent phase may be present in a content ranging from 77% to 99.9% by weight relative to the total weight of the composition.

The make-up and/or cosmetic care compositions according to the invention comprise a binder chosen from compositions comprising an aqueous continuous phase and which may be present in an amount, for example, up to 30% by weight, such as from 0.1% to 23% by weight and further from 3% to 20% by weight, relative to the total weight of the make-up and/or cosmetic care composition.

The aqueous phase of the binder according to the invention may comprise water or a floral water such as cornflower water.

The aqueous phase may also comprise from 0% to 15% by weight, relative to the total weight of the aqueous phase, of a C₂-C₆ lower monoalcohol and/or of a polyol such as glycerol, butylene glycol, isoprene glycol or propylene glycol.

The aqueous phase may also comprise one or more gelling agents, nonlimiting examples of which include hydrophilic polymers such as acrylic acid/ethyl acrylate copolymers and carboxyvinyl polymers. Such polymers or copolymers are, for example, the “carbomers” (CTFA) sold by the company Goodrich under the name Carbopol or the polyglyceryl methacrylate sold by the company Guardian under the name Lubragel or the polyglyceryl acrylate sold under the name Hispagel by the company Hispano Chimica, or, finally, the polyacrylamide/C₁₃-C₁₄ isoparaffin/Laureth 7 mixture sold by the company SEPPIC under the name Sepigel.

Other gelling agents may be used in the invention, of which mention may be made of: polysaccharide derivatives such as cellulose derivatives, for instance carboxymethylcellulose, hydroxymethylcellulose, hydroxypropylcellulose or microcrystalline cellulose; xanthan polymers, gellan polymers, rhamsan polymers, alginates, maltodextrin, starch and its derivatives, hyaluronic acid and its salts, karaya gum, carob flour and guar derivatives, for example hydroxypropylguar.

Polyethylene glycols (PEG) and their derivatives, and polyvinylpyrrolidones and their derivatives may also be used as hydrophilic polymer.

In one embodiment of the invention, the binder comprises an O/W emulsion stabilized with one or more organized systems. For the purposes of the present invention, the expression “organized systems” indicates inverse micelles or “lyotropic liquid crystal” structures which are formed at room temperature by mixing several surfactants or mixing surfactants and polar solvents or mixing several polar solvents, the polar solvents being chosen, for example, from water, glycerol, panthenol, propylene glycol, butylene glycol and mixtures thereof.

The liquid crystal state is an intermediate state between the solid state and the liquid state, often referred to as a mesomorphic state. The organized systems of the present invention are generally thermodynamically stable, and may be chosen from lyotropic liquid crystals. The lyotropic liquid crystal state may be characterized by a person skilled in the art by known means such as transmission electron microscopy (after negative staining or cryofracture) or birefringence under polarized light and X-ray diffraction. The lyotropic liquid crystals may comprise inverse hexagonal liquid crystals, also known as II hexagonal phase or F phase crystals, cubic liquid crystals, also known as I₂ phase crystals, and lamellar liquid crystals, also known as Lₐ phase and D phase crystals.

The lyotropic liquid crystals used in the present invention may be chosen from cubic liquid crystals and lamellar liquid crystals, and mixtures thereof.

In one embodiment of the invention, the binder may comprise an O/W emulsion stabilized with an organized system, for example comprising cubic liquid crystals forming a gel. Thus, the binder may comprise an emulsion stabilized with a dispersion of cubic gel particles.

The term “cubic gel” used in accordance with the present invention denotes transparent gels which are isotropic in polarized light, and are in the form of cubic liquid crystal phases. The cubic liquid crystal phases are organized in a bipolar manner into separate hydrophilic and lipophilic domains forming a thermodynamically stable, three-dimensional network. Such an organization has been described, for example, in “La Recherche”, Vol. 23, pp. 306-315, March 1992 and in “Lipid Technology”, Vol. 2, No. 2, pp.42-45, April 1990, all of which related to such organization is incorporated by reference herein. Depending on the arrangement of the hydrophilic and lipophilic domains, the cubic phase is said to be of normal or reverse type. The term “cubic gel” used in the present invention groups together gels having various types of cubic phases.

Non-limiting representatives of such cubic gels are disclosed, for example, in EP 0 686 386 and EP 0 711 540, all of which disclosure relating to cubic gels is incorporated by reference herein.

In one embodiment of the invention, the binder is chosen from compositions comprising an aqueous continuous phase comprising or, if there is a difference, formed from at least:

(i) 3,7,11,15-tetramethyl-1,2,3-hexadecanetriol, i.e., phytanetriol, wherein said compound is present in an amount, for example, ranging from 0.1% to 15% by weight relative to the total weight of the binder, and

(ii) at least one dispersing and stabilizing agent chosen from surface-active agents chosen from optionally saturated, linear and branched surface-active agents comprising fatty chains having from 8 to 22 carbon atoms, wherein said at least one dispersing and stabilizing agent is present in an amount ranging from 0.1% to 3% by weight relative to the total weight of the binder and wherein said surface-active agents are water-soluble at room temperature.

In one embodiment of the invention, the binder is chosen from compositions comprising cubic gel particles comprising, or if there is a difference, formed from at least phytanetriol and an aqueous phase. The aqueous phase may comprise at least one dispersing and stabilizing agent chosen from surface-active agents chosen from optionally saturated, linear and branched surface-active agents comprising fatty chains having from 8 to 22 carbon atoms.

As a further example, the amount of phytanetriol ranges from 0.5% to 10% by weight relative to the total weight of the binder.

The weight ratio of the phytanetriol to said dispersing and stabilizing agent, as defined above, ranges, for example, from 1:1 to 200:1, such as, for example, from 2:1 to 50:1.

The weight ratio of the phytanetriol to the said dispersing and stabilizing agent, as defined above, may range from 1:1 to 200:1, such as, for example, from 2:1 to 50:1.

Phytanetriol is a known compound which is sold, for example, under the name “Phytanetriol-63926®” by the company ROCHE.

In a further embodiment of the invention, the binder is chosen from compositions comprising:

(1) an aqueous phase, wherein said aqueous phase is present in an amount ranging from 60% to 98% by weight relative to the total weight of said composition; and

(2) an oily phase, wherein said oily phase is present in an amount ranging from 2% to 40% by weight relative to the total weight of said composition, wherein said oily phase is dispersed in said aqueous phase and is stabilized with the aid of cubic gel particles, wherein said cubic gel particles comprise, or if there is a difference, are formed from at least:

(i) at least one compound chosen from 3,7,11,15-tetramethyl-1,2,3-hexadecanetriol, i.e., phytanetriol, N-2-alkoxycarbonyl derivatives of N-methylglucamine, and unsaturated fatty acid monoglycerides, wherein said at least one compound is present in an amount ranging from 0.1% to 15% by weight relative to the total weight of the composition; and

(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging from 0.05% to 3% by weight relative to the total weight of the composition and wherein said surface-active agents are water-soluble at room temperature.

In one embodiment, the weight ratio of said cubic gel particles to said oily phase may range, for example, from 0.02:1 to 1:1, such as, from 0.05:1 to 0.5:1.

According to another embodiment of the invention, the weight ratio of said cubic gel particles to said at least one dispersing and stabilizing agent may range, for example, from 2:1 to 200:1, such as, for example, less than or equal to 50:1.

Non-limiting examples of N-2-alkoxycarbonyl derivatives of N-methylglucamine may be chosen from at least one compound of formula (I):

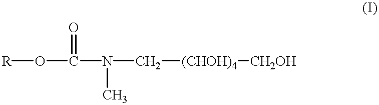

wherein R is chosen from branched (C₆-C₁₈) alkyl groups.

Non-limiting representatives of these derivatives may be chosen from N-2-hexyldecyloxycarbonyl-N-methylglucamine, N-2-ethylhexyloxycarbonyl-N-methylglucamine and N-2-butyloctyloxycarbonyl-N-methylglucamine.

A method for preparing the compounds of formula (I) is disclosed in EP-0 711 540, which preparation method is incorporated by reference herein.

In one embodiment of the invention, the binder is chosen from compositions comprising:

(1) an aqueous phase, wherein said aqueous phase is present in an amount ranging from 60% to 98% by weight relative to the total weight of said composition; and

(2) an oily phase, wherein said oily phase is present in an amount ranging from 2% to 40% by weight relative to the total weight of said composition, wherein said oily phase is dispersed in said aqueous phase and is stabilized with the aid of cubic gel particles, wherein said cubic gel particles comprise or, if there is a difference, are formed from at least:

(i) a mixture comprising:

(a) phytanetriol in an amount ranging from 1% to 40% by weight relative to the total weight of said mixture; and

(b) at least one compound of formula (I):

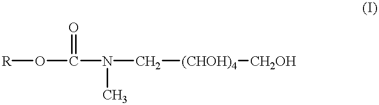

wherein R is chosen from branched (C₆-C₁₈) alkyl groups and said at least one compound of formula (I) is present in an amount ranging from 60% to 99% by weight relative to the total weight of said mixture; and

(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging from 0.05% to 3% by weight relative to the total weight of the composition and wherein said surface-active agents are water-soluble at room temperature.

According to one embodiment component (i) of said cubic gel particles defined above, may comprise, or if there is a difference, be formed from at least a mixture comprising:

(a) phytanetriol in an amount ranging from 10% to 30% by weight relative to the total weight of said mixture; and

(b) at least one compound of formula (I) in an amount ranging from 70% to 90% by weight relative to the total weight of said mixture.

Examples of unsaturated fatty acid monoglycerides are those chosen from (C₁₆-C₂₂) unsaturated fatty chains.

Non-limiting representatives of these monoglycerides may be chosen from glyceryl monooleate, glyceryl monoolein, glyceryl monolinoleate and glyceryl monolinolein.

It is possible to use, in the compositions according to the invention, mixtures comprising at least one compound chosen from unsaturated fatty acid monoglycerides, as defined above, and saturated fatty acid monoglycerides, wherein the proportion of saturated fatty acid monoglycerides is, for example, lower than that of unsaturated fatty acid monoglycerides.

According to another embodiment of the invention, the binder is chosen from compositions comprising:

(1) an aqueous phase, wherein said aqueous phase is present in an amount ranging, for example, from 60% to 98% by weight relative to the total weight of said composition; and

(2) an oily phase, wherein said oily phase is present in an amount ranging, for example, from 2% to 40% by weight relative to the total weight of said composition, and further wherein said oily phase is dispersed in said aqueous phase and is stabilized with the aid of cubic gel particles, wherein said cubic gel particles comprise, or if it makes a difference, are formed from:

(i) a mixture comprising:

(a) phytanetriol in an amount ranging, for example, from 1% to 50%, such as from 10% to 30%, by weight relative to the total weight of said mixture; and

(b) at least one unsaturated fatty acid monoglyceride present in an amount ranging, for example, from 50% to 99%, such as from 70% to 90%, by weight relative to the total weight of said mixture; and

(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging, for example, from 0.05% to 3% by weight relative to the total weight of said composition and further wherein said surface-active agents are water-soluble at room temperature.

Non-limiting examples of said at least one dispersing and stabilizing agent, defined above, may be chosen from:

polyol alkyl ethers, polyol alkenyl ethers, polyol alkyl esters, and polyol alkenyl esters;

N-acylated amino acids and derivatives thereof;

peptides, wherein said peptides are N-acylated with at least one group chosen from alkyl groups and alkenyl groups, and salts thereof;

alkyl ethers, alkenyl ethers, alkyl ester sulfates and alkenyl ester sulfates, derivatives and salts thereof;

polyoxyethylenated alkyl fatty ethers, polyoxyethylenated alkenyl fatty ethers, polyoxyethylenated alkyl fatty esters, and polyoxyethylenated alkenyl fatty esters;

polyoxyethylenated alkyl carboxylic acids, polyoxyethylenated alkenyl carboxylic acids and salts thereof;

N-alkyl betaines, and N-alkenyl betaines; and

alkyltrimethylammonium, alkenyltrimethylammonium and salts thereof.

In the compounds listed above, the alkyl and alkenyl groups have 8 to 22 carbon atoms, and since “at least one” is used, mixtures are manifestly contemplated.

In this embodiment of the invention, the binder has a pH generally ranging, for example, from 5 to 8, such as from 6 to 7.

Binders according to the invention thus prepared are stable when stored for 2 months at a temperature ranging from 4 to 45° C. without showing change in macroscopic or microscopic appearance, color or odor.

The cubic gel particles of the invention may further comprise, or if there is a difference, be formed from, at least one water-insoluble ionic amphiphilic lipid in an amount, for example, ranging from 0.0005% to 5% by weight relative to the total weight of the composition, such as, for further example, from 0.001% to 2% by weight relative to the total weight of the composition.

Non-limiting examples of lipids which may be used in accordance with the invention are chosen from natural, modified or synthetic phospholipids, phosphoric esters of fatty alcohols, N-acylated derivatives of glutamic acid, sodium cetyl sulphate, sodium cocoyl monoglyceride sulphate and quaternary ammonium derivatives and mixtures thereof.

The binder according to the invention, as defined above, may be obtained through fragmentation (with the aid of a homogenizer) of the at least one phytanetriol-based cubic gel, water, and at least one water-soluble surface-active agent comprising optionally saturated, fatty chains, as defined above. Optionally, at least one compound chosen, for example, from water-insoluble ionic amphiphilic lipids and active principles chosen from hydrophilic active principles and lipophilic active principles may be incorporated into the binder.

The cubic gel particles may be obtained by various suitable mechanical means, for example, a high-shear homogenizer of a rotor-stator type such as a “Virtis” homogenizer, and a high-pressure homogenizer operating at a pressure, for example, ranging from 200 to 1 800 bar (20 to 180 MPa).

The average size of the particles in the dispersion according to the invention, as defined above, may, for example, range from 0.05 to 1 μm, such as, for example, less than or equal to 0.5 μm. The particle size of the dispersion may also be modified by varying the nature and concentration of the water-soluble, fatty-chain surface-active agent used.

At this stage in the preparation, it is possible to incorporate into the cubic gel particles of the dispersions as defined above, at least one active principle, such as, for example, hydrophilic active principles and lipophilic active principles.

Needless to say, by virtue of the specific structure of the cubic gel particles, it is possible to incorporate therein both hydrophilic active principles and lipophilic active principles, even though these active principles have a certain level of incompatibility.

The binder according to the invention may thus comprise at least one type of particle chosen from hydrophilic active principles, and lipophilic active principles. Examples of hydrophilic and lipophilic active principles, include compounds such as, hydrophilic and lipophilic UV screening agents.

After formation of the cubic gel particles, said at least one dispersing and stabilizing agent is generally located on the outside of said particles.

In one embodiment of the invention, the binder may further comprise an oily phase, wherein said oily phase optionally comprises at least one compound chosen from lipophilic additive and active principle. These compounds may be incorporated into the dispersion and mechanically stirred with the aid of, for example, a “Virtis®” type homogenizer.

It is thus possible to disperse the oily phase into the continuous aqueous phase in the form of droplets with an average size ranging, for example, from 0.1 micron to 10 microns. This oily phase comprises at least one oil obtained from any source, such as plant origin, animal origin, mineral origin and synthetic origin.

In another embodiment of the invention, the binder may be an O/W emulsion stabilized with an organized system consisting of lamellar lyotropic liquid crystals. The binder is thus a dispersion of vesicles provided with a lamellar liquid crystal coating. Depending on whether these vesicles contain an oily core or an aqueous core, they are commonly referred to as oleosomes or, on the contrary, liposomes and/or niosomes.

Non-limiting examples of dispersions of vesicles comprising an oily core which may be used according to the present invention, are emulsions of oil-in-water type formed from oily globules each provided with a lamellar liquid crystal coating and dispersed in an aqueous phase. Each oily globule can be individually coated with a monolamellar layer or oligolamellar layer obtained from at least one material chosen from lipophilic surfactants, hydrophilic surfactants, fatty acids and ionic amphiphilic lipids. The emulsion can have a pH ranging, for example, from 5.5 to 7.5.

The oily core of these vesicles may be at least one material chosen from oils, lipophilic active agents dissolved in an oil, and active agents dissolved in an oil. The oils which may be used are the oils conventionally used as support in cosmetic compositions, such as, for example, short-chain fatty acid triglycerides, silicone oils, etc.

The expression “oligolamellar layer” indicates a layer comprising from 2 to 5 lipid leaflets. The average size of the coated oily globules is generally, for example, less than 500 nanometers, such as less than 200 nanometers.

In one embodiment of the invention, the lipophilic surfactant sand the hydrophilic surfactants each comprise at least one saturated fatty chain comprising more than about 12 carbon atoms, such as, for example, from 16 to 22 carbon atoms.

In another embodiment of the invention, the lipophilic surfactant has an HLB ranging from about 2 to about 5. The term HLB (hydrophilic-lipophilic balance) is known in the art to indicate the balance between the size and strength of the hydrophilic group and the size and strength of the lipophilic group of the surfactant.

For example, such lipophilic surfactants may be chosen from sucrose distearate, diglyceryl distearate, tetraglyceryl tristearate, decaglyceryl decastearate, diglyceryl monostearate, hexaglyceryl tristearate, decaglyceryl pentastearate, sorbitan monostearate, sorbitan tristearate, diethylene glycol monostearate, the glyceryl ester of palmitic acid and of stearic acid, 2 EO polyoxyethylenated monostearate (comprising 2 oxyethylene units), glyceryl monobehenate and glyceryl dibehenate, pentaerythrityl tetrastearate, and, of course, mixtures thereof.

The hydrophilic surfactant may have an HLB ranging, for example, from about 8 to about 12.

Non-limiting examples of such hydrophilic surfactants include 4 EO polyoxyethylenated sorbitan monostearate, 20 EO polyoxyethylenated sorbitan tristearate, 8 EO polyoxyethylenated monostearate, hexaglyceryl monostearate, 10 EO polyoxyethylenated monostearate and 12 EO polyoxyethylenated distearate, and 20 EO polyoxyethylenated methylglucose distearate.

The fatty acids used in the context of the present invention may be, for example, saturated fatty acids comprising from 16 to 22 carbon atoms. For example, palmitic acid, stearic acid, arachidic acid and behenic acid may be used.

The ionic amphiphilic lipid used in the context of the present invention may be, for example, chosen from neutralized anionic lipids, amphoteric lipids and alkylsulphonic derivatives, and mixtures thereof.

The neutralized anionic lipids may be, for example, chosen from:

alkali metal salts of dicetyl phosphate, such as the sodium and potassium salts;

alkali metal salts of dimyristyl phosphate, such as the sodium and potassium salts;

alkali metal salts of cholesteryl sulphate, such as the sodium salt;

alkali metal salts of cholesteryl phosphate, such as the sodium salt;

monosodium salts of acylglutamic acids and disodium salts of acylglutamic acids, such as the monosodium salts of N-stearoylglutamic acid and the disodium salts of N-stearoylglutamic acid, and

the sodium salt of phosphatidic acid.

The amphoteric lipids may be, for example, chosen from phospholipids. such as pure soybean phosphatidylethanolamine.

The alkylsulphonic derivatives may be, for example, chosen from the compounds of formula:

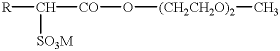

wherein R is at least one group chosen from C₁₆H₃₃ and C₁₈H₃₇ groups and M is chosen from alkali metals, such as sodium.

In an embodiment of the invention, at least one coating material may be chosen from hydrophilic surfactants, lipophilic surfactants, fatty acids and ionic amphiphilic lipids, and the amount of coating material for the oily globules may comprise, for example, about 2% to about 6% by weight, relative to the total weight of the binder. As a further example, this amount of coating material may range from 3% to 4%. The relative amounts of 1) lipophilic surfactant, 2) hydrophilic surfactant and 3) fatty acid or, alternatively, ionic amphiphilic lipid may vary within the following respective ranges: 35-55%/25-40%/15-35% by weight relative to the total weight of the coating material. As mentioned above, each oily globule is coated with either a monolamellar layer or an oligolamellar layer.

The fatty phase, that is to say the coated oily droplets, is present in an amount, for example, from 5% to 50%, as a further example, from 10% to 40%, by weight relative to the total weight of the binder.

The ratio of the weight of the oily globules to the weight of the constituent elements of the coating ranges, for example, from 2:1 to 13:1; as a further example, this ratio is about 7:1.

When a fatty acid is used in the preparation of these vesicles, the aqueous phase of the binder may comprise a basic agent for neutralizing the fatty acid present in the oily phase. This basic agent should thus be present in an amount at least equal to that required to neutralize all of the fatty acid. Basic agents which may be used, for example, include sodium hydroxide, triethanolamine, lysine, and arginine.

Such dispersions, and the process for preparing them, are disclosed in documents EP-A-0 641 557 and EP-A-0 705 593, the portions of which relating to such dispersions and ways of preparing them are incorporated by reference herein.

The vesicles comprising an aqueous core provided with a lamellar liquid crystal coating which may be used according to the present invention may be obtained, for example, from at least one amphiphilic lipid having the general formula:

X-Y

wherein X is a hydrophilic group and Y is a lipophilic group. The amphiphilic lipids may be ionic lipids, for which the group X is ionic, or nonionic lipids for which the group X is nonionic. Mixtures of ionic amphiphilic lipids, mixtures of nonionic amphiphilic lipids and mixtures of these two types of lipid may be used to manufacture the vesicles.

Representative vesicle dispersions which may be used for the present invention are disclosed, for example, in documents FR-A-2 315 991, FR-A-2 408 387 and FR-A-2 485 921, the portions of which relating to such dispersions are incorporated by reference herein.

Representative examples of vesicle dispersions which may be used according to the present invention include aqueous dispersions of vesicles comprising a lipid-phase membrane encapsulating an aqueous phase. The lipid phase may comprise at least one nonionic amphiphilic lipid and at least one ionic amphiphilic lipid wherein:

the at least one nonionic amphiphilic lipid is chosen from esters formed from (a) at least one polyol chosen polyethylene glycol bearing from 1 to 60 ethylene oxide units, sorbitan, sorbitan bearing 2 to 60 ethylene oxide units, glycerol bearing 2 to 30 ethylene oxide units, polyglycerols comprising 2 to 15 glycerol units, sucroses, glucoses bearing 2 to 30 ethylene oxide units, and (b) at least one fatty acid comprising an alkyl chain chosen from linear and branched, saturated and unsaturated C5-C17 alkyl chains, the number of alkyl chains per polyol group ranging from 1 to 10; and

the at least one ionic amphiphilic lipid is chosen from ionic amphiphilic lipids giving the dispersion a pH ranging from 5.5 to 7.5;

the weight ratio of the at least one nonionic amphiphilic lipid to the at least one ionic amphiphilic lipid in the lipid phase ranging from 50:1 to 50:25 and the weight ratio of the lipid phase to the aqueous phase of the dispersion ranging from 1:1 000 to 300:1 000.

The at least one ionic amphiphilic lipid combined with the at least one nonionic amphiphilic lipid is, for example, chosen from:

alkali metal salts of dicetyl phosphate and alkali metal salts of dimyristyl phosphate;

alkali metal salts of cholesteryl sulphate;

alkali metal salts of cholesteryl phosphate;

lipoamino acids and salts thereof, such as monosodium acylglutamates and disodium acylglutamates, for example the disodium salt of N-stearoyl-L-glutamic acid sold under the name Acylglutamate HS21 by the company Ajinomoto;

the sodium salts of phosphatidic acid;

phospholipids;

alkylsulphonic derivatives of the formula:

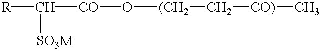

wherein R is chosen from C₁₆-C₂₂ alkyl groups, such as C₁₆H₃₃ and C₁₈H₃₇ groups and mixtures thereof, and M is chosen from alkali metals and alkaline-earth metals, such as sodium, and

mixtures thereof.

It is known in the art to incorporate into the lipid phase constituting the lipid membrane of the vesicles at least one additive whose main function is to reduce the permeability of the vesicles, to prevent them from flocculating and melting and to increase their degree of encapsulation. According to the invention, at least one additive may be added to the lipid phase. This at least one additive may be, for example, chosen from:

sterols, such as phtyosterols and cholesterol,

C₁₄ to C₃₀ alcohols and diols, and

C₁₄ to C₃₀ amines and quaternary ammonium derivatives thereof,

The vesicles of this embodiment of the invention generally have a mean diameter ranging, for example, from 10 to 5,000 nm. When the aqueous phase comprises a dispersion of water-immiscible liquid droplets, these droplets advantageously have a mean diameter ranging from 100 to 10,000 nm.

It is also known in the art to produce vesicles which contain at least one active compounds. If the at least one active compound is water-soluble, it may be introduced into the encapsulated aqueous phase of the vesicles. If the at least one active compound is liposoluble, it may be introduced into the lipid phase constituting the membrane. If the at least one active compound is amphiphilic, it may be distributed between the lipid phase and the encapsulated aqueous phase, with a partition coefficient which varies according to the nature of the amphiphilic active compound and the respective compositions of the lipid phase and of the encapsulated aqueous phase.

The aqueous phase of the binder of this embodiment of the invention may also contain a dispersion of droplets of a conventional oily phase.

Such dispersions and the process for preparing them are disclosed in document EP-A-0 582 503, the portion of which relating to the dispersions and preparation processes being incorporated by reference herein.

Dispersions which may be used according to the present invention may also be emulsions of an oily phase in an aqueous phase comprising oil globules with a diameter of less than 500 nanometers, the emulsion comprising ionic polymer particles, the ratio of the amount of polymer particles to the amount of oily phase ranging from 1:5 to 1:40. Such emulsions and a process for preparing them are disclosed in document EP-A-0 864 320, the relevant part of which is incorporated by reference.

The binder of the present invention may also be in the form of any oil-in-water dispersion, any simple O/W or multiple W/O/W emulsion or in the form of a nanoemulsion.

The binder may also comprise at least one surfactant with an HLB (hydrophilic-lipophilic balance) of greater than or equal to 8. This surfactant may have a minimal reduction of the surface tension of water at the critical micelle concentration of the surfactant; thus, its surface tension γ_{surf}, measured according to the conventional methods known to those skilled in the art, can be greater than or equal to 50 mN/m.

Non-limiting examples of surfactants which may be used according to the present invention include: stearic acid combined with triethanolamine, the mixture of glyceryl monostearate and distearate, sodium lauroyl sarcosinate, cetearyl glucoside, PEG-40 stearate, sorbitan tristearate, sorbitan stearate, polysorbate-60, the mixture sorbitan stearate/sucrose cocoate, the mixture glyceryl stearate/PEG-100 stearate, PEG-400, glyceryl stearate, the mixture PEG-6/PEG-32/glycol stearate, and mixtures thereof.

The oily phase of the binders according to the invention may comprise any oily compound conventionally used in emulsions.

Non-limiting representative oils which may be used in accordance with the invention may be chosen from mink oil, turtle oil, soybean oil, grape pip oil, sesame oil, corn oil, rapeseed oil, sunflower oil, cotton oil, avocado oil, olive oil, castor oil, jojoba oil and groundnut oil; hydrocarbon oils, such as liquid paraffin, squalene and petroleum jelly; fatty esters, such as isopropyl myristate, isopropyl palmitate, butyl stearate, hexyl laurate, isononyl isononate, 2-ethylhexyl palmitate, 2-hexyidecyl laurate, 2-octyldecyl palmitate, 2-octyldodecyl myristate, 2-octyldodecyl lactate, 2-diethylhexyl succinate, diisostearyl malate, glyceryl triisostearate, diglyceryl triisostearate; perfluoro oils; higher fatty acids, such as myristic acid, palmitic acid, stearic acid, behenic acid, oleic acid, linoleic acid, linolenic acid and isostearic acid; higher fatty alcohols, such as cetanol, stearyl alcohol and oleyl alcohol; silicone oils, for instance polydimethylsiloxanes, polyalkylmethylsiloxanes and for example poly(C₁-C₂₀)alkylsiloxanes, for instance phenylsilicone oils and cetyidimethicone (CTFA name), cyclocopolymers such as dimethylsiloxane/methylalkylsiloxane, organomodified polydimethylsiloxanes, for instance oxyethylenated oxypropylenated polydimethyldisiloxanes, and mixtures thereof.

The binder may further comprise at least one fatty substance other than those already mentioned, chosen from waxes and pasty fatty substances.

The pasty fatty compounds may be defined with the aid of at least one physicochemical property chosen from:

a viscosity ranging from 0.1 to 40 Pa.s (1 to 400 poises), such as, for example, from 0.5 to 25 Pa.s, measured at 40° C. with a Contraves TV rotary viscometer equipped with an MS-r3 or MS-r4 spindle, at a frequency of 240 rpm, and

a melting point ranging from 25 to 70° C., such as, for example, from 25 to 55° C.

Waxes which may be used in the compositions of the invention, may, for example, be chosen from beeswaxes, lanolin waxes, Chinese insect waxes, carnauba wax, candelilla wax, ouricury wax, cork fiber waxes, sugar cane waxes, Japan waxes, hydrogenated jojoba waxes, hydrogenated oils, such as, for example, hydrogenated sunflower oil, hydrogenated castor oil, hydrogenated coconut oil and hydrogenated lanolin, paraffins, microcrystalline waxes, Montan waxes, ozokerites, polyethylene waxes, waxes obtained by Fischer-Tropsch synthesis, waxy copolymers and waxy esters thereof, and silicone waxes, such as, for example, polyalkoxysiloxanes, polyalkylsiloxanes, and mixtures thereof.

The binder may optionally further comprise a volatile portion such as, for example, at least one volatile oil.

The term “volatile oil” indicates any compound capable of evaporating upon contact with the skin. Oils having a flashpoint high enough to allow these oils to be used in formulation, and low enough to obtain the desired evanescent effect may be chosen. In one embodiment of the invention, oils with flashpoints ranging from 40 to 100° C. are chosen.

Non-limiting examples of these volatile compounds may be chosen from hydrocarbon-based oils, such as, for example, isoparaffins such as, isododecane, cyclic volatile silicones comprising from 3 to 8 silicon atoms, such as, 4 to 6 silicon atoms, for example, chosen from cyclotetradimethylsiloxane, cyclopentadimethylsiloxane and cyclohexadimethylsiloxane, such as, the products sold under the names “DC Fluid 244”, “DC Fluid 245”, “DC Fluid 344” and “DC Fluid 345” by the company Dow Corning, linear volatile silicones comprising from 2 to 9 silicon atoms, for example hexamethyldisiloxane, hexylheptamethyltrisiloxane and octylheptamethyltrisiloxane.

As mentioned above for the various embodiments of the invention, the binder may also comprise active agents. Non-limiting examples of various active principles may be chosen from:

antioxidants and free-radical scavengers chosen from proteins, enzymes, lactoperoxidase, lactoferrin, peptides and derivatives thereof, sequestrants, flavonoids, chlorophylline, ethoxyquine, guanosine, tocopherols and derivatives thereof, ascorbyl palmitate, β-carotene, vitamin E and derivatives thereof, vitamin C and derivatives thereof and vitamin A and derivatives thereof,

moisturizing and wetting agents chosen from hyaluronic acid, and its sodium salt, β-glycerophosphate, glycerol, sorbitol and panthenol,

UV screening agents chosen from the products sold under the names “Eusolex 232®” by the company Merck, “Parsol 1789®” and “Parsol MCX®” by the company Givaudan-Rouré, “Mexoryl SX®” by the company Chimex and “Uvinul T150®” by the company BASF,

keratolytic agents chosen from proteolytic enzymes, salicylic acid and derivatives thereof, such as 5-n-dodecanoylsalicylic acid, and retinoic acid and derivatives thereof,

tanning accelerators chosen from caffeine, and tyrosine derivatives, such as, glucose tyrosinate and the disodium salt of N-L-malyltyrosine,

depigmenting agents chosen from kojic acid, glycolic acid, vitamin C, magnesium ascorbyl phosphate, arbutin, and derivatives thereof,

natural colorants chosen from dyestuffs extracted from plants, for example, chlorophylline and β-carotene, and dyestuffs extracted from animals, for example, cochineal carmine, and caramel,

self-tanning agents chosen from dihydroxyacetone and indoles,

liporegulators chosen from γ-orizanol, extract of Centella asiatica comprising genin and asiatic acid, caffeine and theophylline,

anti-ageing and anti-wrinkle agents chosen from hydroxy acids, for example, glycolic acid, n-octanoyl salicylic acid, retinol and derivatives thereof, such as, retinyl acetate, palmitate, propionate, and retinoids,

antiinflammatories and cicatrizing agents chosen from α-bisabolol, corticoids, 18-β-glycyrrhetinic acid and its salts such as, for example, its ammonium salt, extract of Centella asiatica, and aloe vera,

antibacterial and antifungal agents chosen from benzalkonium chloride, chlorhexidine, hexetidine and hexamidine,

insect repellents, chosen from diethyltoluamides and dimethyltoluamides,

deodorants chosen from hexachlorophene, and the product Triclosan sold under the name “Irgasan DP 300®” by the company Ciba Geigy,

antidandruff agents chosen from octopirox, and pyridinethione derivatives such as those sold under the name “Omadine®” by the company Olin,

agents for preventing hair loss, chosen from methyinicotinate, hexylnicotinate, and Minoxidil,

hair dyes chosen from oxidation bases, oxidation couplers, direct dyes and self-oxidizing dyes,

permanent-wave reducing agents chosen from thioglycolic acid, cysteine, cysteamine, n-acetylcysteine, N-acetylcysteamine and glyceryl thioglycolate, and

skin and hair conditioners chosen from cationic polymers and cations.

The composition of the invention may also comprise any additive commonly used in the field under consideration, such as antioxidants, essential oils, preserving agents, neutralizing agents, W/O and O/W surfactants, vitamins and anti-wrinkle active agents.

Needless to say, a person skilled in the art will take care to select this or these optional additional compounds, and/or the amount thereof, wherein the advantageous properties of the composition according to the invention are not, or are not substantially, adversely affected by the addition envisaged.

The composition according to the invention may be in the form of a powder, for example a compact, free, pressed or cast powder. In the case of a free powder, the binder may be, for example, present in an amount up to 15% by weight relative to the total weight of the composition, such as from 1% to 5% by weight relative to the total weight of the composition. For a compact powder, the binder content may be, for example, present in an amount from 1% to 30% by weight, such as from 5% to 20% by weight relative to the total weight of the composition.

The compositions according to the invention may be prepared by the known methods for preparing cosmetic powders.

One process for preparing the compositions according to the present invention comprises the following steps:

a) mixing together the pulverulent compounds,

b) grinding the mixture obtained in a) into fine particles,

c) adding the binder and mixing until a homogeneous composition is obtained.

The mixing in step a) may be carried out, for example, using a turbomixer such as a “Baker-Perkins” machine. The grinding in step b) may be carried out using a toothed roll crusher or an air-jet mill. The addition of the binder may be carried out dropwise with stirring in a mixer whose temperature can be kept constant at 35° C.

The invention is illustrated in greater detail in the examples which follow.

In the examples which follow, the amounts are given as percentages by weight relative to the total weight of the composition.

### EXAMPLE 1

The following compositions were prepared in the form of powders (the amounts are given as percentages by weight relative to the total weight of the composition):

| | |
|---|---|
| Phase A: | |
| talc | X% |
| iron oxides | 2.74% |
| Nylon powder | 10% |
| titanium oxide | 1% |
| preserving agent | 0.2% |
| Phase B: | |
| binder (dispersion of cubic gel particles) | Y% |

with:

| | X % | Y % |
|---|---|---|
| Composition 1 | 77.06 | 9 |
| Composition 2 | 74.06 | 12 |
| Composition 3 | 71.06 | 15 |

The composition of the binder (dispersion of cubic gel particles) was as follows (the amounts are given as percentages by weight relative to the total weight of the composition):

| | |
|---|---|
| isopropyl myristate | 1.64% |
| castor oil | 2.46% |
| liquid petroleum jelly | 12.36% |
| liquid lanolin | 1.26% |
| water | 70.95% |
| imidazolinylurea | 0.3% |
| glycerol | 5% |
| monosodium salt of N-stearoyl-L-glutamic acid | 0.03% |
| phytanetriol | 2.97% |
| petroleum jelly | 2.28% |
| chlorphenesine | 0.25% |
| oxyethylenated sorbitan monopalmitate (40 EO) | 0.5% |

This binder was prepared in the following manner: an aqueous dispersion of cubic gel particles was obtained by mixing together 2.97 g of phytanetriol and 0.03 g of monosodium salt of N-stearoyl-L-glutamic acid and 1.28 g of water, to which were added 75.17 g of an aqueous solution comprising 0.5 g of oxyethylenated sorbitan monopalmitate and 5 g of glycerol. The mixture was then predispersed, after which it was homogenized at room temperature, using a “Virtis®” homogenizer at 35,000 rpm for 5 minutes, this stirring being repeated 4 times.

The preserving agents (chlorphenesine and imidazolinylurea) and the fatty substances (isopropyl myristate, castor oil, liquid petroleum jelly, liquid lanolin and petroleum jelly) were then added to the aqueous dispersion of cubic gel particles obtained. After stirring at room temperature using a Virtis® homogenizer at 35,000 rpm for 5 minutes, this stirring being repeated 5 times, a stable, homogeneous dispersion was obtained.

For each composition 1 to 3, a mixture of the compounds of phase A was first prepared in a turbomixer (such as a “Baker-Perkins” turbomixer). This mixture was then ground using a toothed roll crusher or an air-jet mill. Finally, the binder (phase B) was added dropwise to phase A with stirring in a turbomixer (such as a Baker-Perkins turbomixer), the temperature of which was kept constant at 35° C. The two phases were mixed together until a homogeneous product was obtained. Powders with good cohesion of the pulverulent compounds were obtained.

These powders gave a fresh sensation when applied to the skin.

### EXAMPLE 2

The following compositions were prepared in the form of powders (the amounts are given as percentages by weight relative to the total weight of the composition):

| | |
|---|---|
| Phase A: | |
| talc | X % |
| iron oxides | 2.74% |
| Nylon powder | 10% |
| titanium oxide | 1% |
| preserving agent | 0.2% |
| Phase B: | |
| binder (dispersion of vesicles with | Y % |
| an oily core, having a lamellar liquid | |
| crystal coating) | |

with:

| | X % | Y % |
|---|---|---|
| Composition 4 | 77.06 | 9 |
| Composition 5 | 74.06 | 12 |
| Composition 6 | 71.06 | 15 |

The composition of the binder (dispersion of vesicles with an oily core, having a lamellar liquid crystal coating) was as follows (the amounts are given as percentages by weight relative to the total weight of the composition):

| Phase 1 | |
|---|---|
| polyglyceryl distearate | 2% |
| polyethylene glycol monostearate | 1.35% |
| stearic acid | 1% |
| petroleum jelly | 25.62% |
| castor oil | 4.305% |
| isopropyl myristate | 2.87% |
| lanolin | 2.205% |
| Phase 2 | |
| water | 53.9% |
| triethanolamine | 0.25% |
| glycerol | 5% |
| 2-phenoxyethanol | 1% |
| chlorphenesine | 0.25% |
| phenylethyl alcohol | 0.25% |

Preparation of the Binder:

Phase 1 and phase 2 were brought separately to a temperature of 70° C. Phase 2 was introduced rapidly into phase 1 with vigorous stirring (rotor-stator). The stirring and temperature were maintained for 10 minutes. This emulsion was then treated in a high-pressure homogenizer at 500 bar, 3 times in succession, and then cooled to room temperature. The dispersion obtained was fluid, milky and had a particle size of less than 500 nm, of about 220 nm.

For each composition 4 to 6, a mixture of the compounds of phase A was first prepared in a turbomixer (such as a “Baker-Perkins” turbomixer). This mixture was then ground using a toothed roll crusher or an air-jet mill. Finally, the binder (phase B) was added dropwise to phase A with stirring in a turbomixer (such as a Baker-Perkins turbomixer), the temperature of which was kept constant at 35° C. The two phases were mixed together until a homogeneous product was obtained. Powders with good cohesion of the pulverulent compounds were obtained.

These powders gave a fresh sensation when applied to the skin.

### EXAMPLE 3

### Comparative Example

The following composition was prepared in the form of a powder according to the prior art:

Composition 7:

| Phase A: | |
|---|---|
| talc | 73.8% |
| yellow iron oxide | 1.6% |
| red iron oxide | 1.7% |
| black iron oxide | 0.5% |
| Nylon powder | 10% |
| nanotitanium oxide | 3% |
| methylsilsequioxane resin | 5.0% |
| methyl p-hydroxybenzoate | 0.3% |
| hydrated magnesium carbonate | 0.2% |
| fragrance | 0.2% |
| Phase B: Binder | |
| polymethylcetyldimethylsiloxane | 0.45% |
| mixture of trimethyl siloxysilicate | |
| resin and of polydimethylsiloxane | 0.73% |
| polydimethylsiloxane | 2.52% |

Composition 7 thus comprised a conventional binder based on silicone compounds and comprised no aqueous phase.

Composition 8 below was prepared according to the invention:

Composition 8:

| | |
|---|---|
| Phase A: | |
| talc | 73.8% |
| yellow iron oxide | 1.6% |
| red iron oxide | 1.7% |
| black iron oxide | 0.5% |
| Nylon powder | 10% |
| nanotitanium oxide | 3% |
| methylsilsesquioxane resin | 5.0% |
| methyl p-hydroxybenzoate | 0.3% |
| hydrated magnesium carbonate | 0.2% |
| fragrance | 0.2% |
| Phase B: | |
| binder (dispersion of vesicles with a | 3.7% |
| lamellar liquid crystal coating) | |

The composition of the binder in composition 8 corresponded to that of Example 2 mentioned above.

Compositions 7 and 8 were prepared according to the conventional methods. The compounds of phase A were mixed together in a band mixer. The binder (phase B) was introduced dropwise. Phases A and B were mixed together until a homogeneous product was obtained. This product was then ground using an air-jet mill. The powder obtained was screened and compacted.

The calorimetric parameters were measured on the compact powders using a Minolta CR-300 Chromameter colorimeter (technical parameters: measurement geometry: d/0°, size of the measuring area: 8 mm, source: xenon arc lamp, illuminant D65).

The results are expressed in the (L, a, b) system in which L represents the luminance, a represents the red-green axis (−a=green, +a=red) and b represents the yellow-blue axis (−b=blue, +b=yellow). They are also expressed in the (C, h) system, the latter representing the polar coordinates of the Cartesian coordinates (a, b). Thus, C is the radius and is defined by the following relationship:

C=\sqrt{a²+b²}

C thus represents the “saturation” of the color, i.e. its brightness, and h represents the “shade angle” of the color.

| Colorimetric | | | |
|---|---|---|---|
| parameters | Composition 7 | Composition 8 | Δ |
| L | 63.92 ± 1.63 | 65.74 ± 0.14 | 1.82 |
| A | +12.04 ± 0.25 | +13.81 ± 0.06 | 1.77 |
| B | +18.33 ± 0.78 | +21.32 ± 0.14 | 2.99 |
| C | 21.9 | 25.5 | 4.6 |
| H | 56.7 | 56.9 | 0.2 |

Compositions 7 and 8 had substantially the same shade angle h. This indicates that the color shade does not change from one composition to another. On the other hand, for the composition 8 whose binder is an O/W emulsion stabilized with vesicles with an oily core comprising a lamellar phase, and which is thus in accordance with the present invention, a significant increase in the calorimetric parameters a, b and thus c was observed when compared with composition 7, the binder of which was anhydrous. This shows that the color saturation, that is to say its brightness, is markedly greater for a composition comprising a binder with an aqueous continuous phase in accordance with the present invention than for a composition comprising an anhydrous binder such as a mixture of silicone compounds.

Thus, it is possible, by virtue of the present invention, to prepare compositions comprising a smaller amount of pigments than the compositions of the prior art, while at the same time obtaining the same intensity of color. Similarly, by using the same amount of pigments as a composition of the prior art, the use of the binder according to the invention makes it possible to obtain a composition whose color is much brighter, more intense and more developed.

What is claimed is:

1. A make-up and/or cosmetic care composition in the form of a powder comprising a pulverulent phase and a binder, wherein the binder is chosen from compositions comprising an aqueous continuous phase in said powder composition.

2. A composition according to claim 1, wherein the pulverulent phase comprises at least one material chosen from pigments, nacres, fillers, and flakes.

3. A composition according to claim 2, wherein the pigments are chosen from titanium dioxide, zirconium dioxide, cerium dioxide, zinc oxide, iron oxide, chromium oxide, nanotitaniums, nanozincs, ferric blue, carbon black, lakes, acidic dyes, azo dyes, anthraquinone dyes, pigments coated with silicone compounds, and pigments coated with polymers.

4. A composition according to claim 2, wherein the pulverulent phase comprises pigments present in a content ranging from 0.05% to 80% by weight relative to the total weight of the composition.

5. A composition according to claim 4, wherein the pigments are present in a content ranging from 0.5% to 50% by weight relative to the total weight of the composition.

6. A composition according to claim 2, wherein the fillers are chosen from talc, mica, silica, kaolin, Nylon powder, poly-β-alanine powder, polyethylene powder, Teflon, lauroyllysine, starch, boron nitride, bismuth oxychloride, tetrafluoroethylene polymer powders, polymethyl methacrylate powders, polyurethane powders, polystyrene powders, polyester powders, synthetic hollow microspheres, microsponges, silicone resin microbeads, zinc oxide, titanium oxide, zirconium oxide, cerium oxide, precipitated calcium carbonate, magnesium carbonate, magnesium hydrocarbonate, hydroxyapatite, hollow silica microspheres, glass microcapsules, ceramic microcapsules, and metal soaps derived from organic carboxylic acids comprising from 8 to 22 carbon atoms.

7. A composition according to claim 6 wherein said organic carboxylic acids comprise from 12 to 18 carbon atoms.

8. A composition according to claim 2 wherein the pulverulent phase comprises fillers present in a content ranging from 0.1% to 99.9% by weight relative to the total weight of the composition.

9. A composition according to claim 8 wherein the fillers are present in a content ranging from 70% to 99.9% by weight relative to the total weight of the composition.

10. A composition according to claim 2, wherein the pulverulent phase comprises nacres present in a content ranging from 0.05% to 80% by weight relative to the total weight of the composition.

11. A composition according to claim 10, wherein the nacres are present in a content ranging from 2% to 50% by weight relative to the total weight of the composition.

12. A composition according to claim 1, wherein the pulverulent phase is present in a content of at least 50% by weight relative to the total weight of the composition.

13. A composition according to claim 12, wherein the pulverulent phase is present in a content of at least 70% by weight relative to the total weight of the composition.

14. A composition according to claim 1, wherein the binder is present in an amount ranging up to 30% by weight, relative to the total weight of the composition.

15. A composition according to claim 14, wherein the binder is present in an amount rangining from 0.1% to 23% by weight, relative to the total weight of the composition.

16. A composition according to claim 1, wherein the aqueous phase of the binder comprises at least one substance chosen from water and floral water.

17. A composition according to claim 1, wherein the aqueous phase comprises from 0% to 15% by weight, relative to the total weight of the aqueous phase, of at least one substance chosen from C₂-C₆ lower monoalcohols and polyols.

18. A composition according to claim 1, wherein the aqueous phase comprises at least one gelling agent.

19. A composition according to claim 18, wherein said at least one gelling agent is chosen from hydrophilic polymers, polysaccharide derivatives, xanthan polymers, gellan polymers, rhamsan polymers, alginates, maltodextrin, starch and its derivatives, hyaluronic acid and its salts, karaya gum, carob flour, and guar derivatives.

20. A composition according to claim 1, wherein the binder is an O/W emulsion stabilized with at least one organized system.

21. A composition according to claim 20, wherein said at least one organized system is chosen from lyotropic liquid crystals.

22. A composition according to claim 21, wherein said lyotropic liquid crystals are chosen from cubic liquid crystals and lamellar liquid crystals.

23. A composition according to claim 20, wherein said at least one organized system comprises cubic liquid crystals forming a gel.

24. A composition according to claim 1, wherein said aqueous continuous phase comprises:
(i) phytanetriol, wherein said phytanetriol is present in an amount ranging from 0.1% to 15% by weight relative to the total weight of the binder, and
(ii) at least one dispersing and stabilizing agent chosen from surface-active agents chosen from optionally saturated, linear and branched surface active agents comprising fatty chains having from 8 to 22 carbon atoms, wherein said at least one dispersing and stabilizing agent is present in an amount ranging from 0.1% to 3% by weight relative to the total weight of said binder and further wherein said surface-active agents are water-soluble at room temperature.

25. A composition according to claim 24, wherein said phytanetriol is present in an amount ranging from 0.5% to 10% by weight relative to the total weight of the binder.

26. A composition according to claim 25, wherein the weight ratio of said phytanetriol to said at least one dispersing and stabilizing agent ranges from 2:1 to 200:1.

27. A composition according to claim 26, wherein the weight ratio of the phytanetriol to the dispersing and stabilizing agent ranges from 2:1 to 50:1.

28. A composition according to claim 1, wherein said binder comprises:
(1) an aqueous continuous phase, wherein said aqueous phase is present in an amount ranging from 60% to 98% by weight relative to the total weight of said composition; and
(2) an oily phase, wherein said oily phase is present in an amount ranging from 2% to 40% by weight relative to the total weight of said composition, and further wherein said oily phase is dispersed in said aqueous phase and is stabilized with the aid of cubic gel particles, said cubic gel particles being formed from at least:
(i) at least one compound chosen from phytanetriol, N-2-alkoxycarbonyl derivatives of N-methylglucamine, and unsaturated fatty acid monoglycerides, wherein said at least one compound is present in an amount ranging from 0.1% to 15% by weight relative to the total weight of the composition; and
(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging from 0.05% to 3% by weight relative to the total weight of said composition and further wherein said surface-active agents are water-soluble at room temperature.

29. A composition according to claim 28, wherein the weight ratio of said cubic gel particles to said oily phase ranges from 0.02:1 to 1:1.

30. A composition according to claim 29, wherein the weight ratio of said cubic gel particles to said oily phase ranges from 0.05:1 to 0.5:1.

31. A composition according to claim 28, wherein the weight ratio of said cubic gel particles to said at least one dispersing and stabilizing agent ranges from 2:1 to 200:1.

32. A composition according to claim 28, wherein the weight ratio of said cubic gel particles to said at least one dispersing and stabilizing agent is less than or equal to 50:1.

33. A composition according to claim 28, wherein the N-2-alkoxycarboxyl derivatives of N-methylglucamine correspond to the following formula (I): 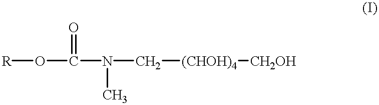
in which:
R is chosen from branched C₆-C₁₈ alkyl radicals.

34. A composition according to claim 33, wherein the derivative is chosen from N-2-hexyldecyloxycarbonyl-N-methylglucamine, N-2-ethylhexyloxycarbonyl-N-methylglucamine, and N-2-butyloctyloxycarbonyl-N- methylglucamine.

35. A make-up and/or cosmetic care composition in powder form comprising a pulverulent phase and a binder in said powder composition, wherein said binder is chosen from compositions comprising:
(1) an aqueous phase, wherein said aqueous phase is present in an amount ranging from 60% to 98% by weight relative to the total weight of said composition; and
(2) an oily phase, wherein said oily phase is present in an amount ranging from 2% to 40% by weight relative to the total weight of said composition, and further wherein said oily phase is dispersed in said aqueous phase and is stabilized with the aid of cubic gel particles, said cubic gel particles being formed from at least:
(i) a mixture comprising:
(a) phytanetriol in an amount ranging from 1% to 40% by weight relative to the total weight of said mixture; and
(b) at least one compound of formula (I): 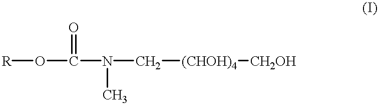
wherein R is chosen from branched (C₆-C₁₈) alkyl groups and said at least one compound of formula (I) is present in an amount ranging from 60% to 99% by weight relative to the total weight of said mixture; and
(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging from 0.05% to 3% by weight relative to the total weight of said composition and further wherein said surface-active agents are water-soluble at room temperature.

36. A composition according to claim 35, wherein said mixture comprises:
(a) phytanetriol in an amount ranging from 10% to 30% by weight relative to the total weight of said mixture; and
(b) at least one compound of formula (I) in an amount ranging from 70% to 90% by weight relative to the total weight of said mixture.

37. A composition according to claim 28, wherein the unsaturated fatty acid monoglycerides comprise a C₁₆-C₂₂ unsaturated fatty chain.

38. A composition according to claim 37, wherein the unsaturated fatty acid monoglycerides are chosen from glyceryl monooleate, glyceryl monoolein, glyceryl monolinoleate, and glyceryl monolinolein.

39. A make-up and/or cosmetic care composition in powder form comprising a pulverulent phase and a binder in said powder composition, wherein said binder is chosen from compositions comprising:
(1) an aqueous phase, wherein said aqueous phase is present in an amount ranging from 60% to 98% by weight relative to the total weight of said composition; and
(2) an oily phase, wherein said oily phase is present in an amount ranging from 2% to 40% by weight relative to the total weight of said composition, and further wherein said oily phase is dispersed in said aqueous phase and is stabilized with the aid of cubic gel particles, said cubic gel particles being formed from at least:
(i) a mixture comprising:
(a) phytanetriol in an amount ranging from 1% to 50% by weight relative to the total weight of said mixture; and
(b) at least one unsaturated fatty acid monoglyceride present in an amount ranging from 50% to 99% by weight relative to the total weight of said mixture; and
(ii) at least one dispersing and stabilizing agent chosen from surface-active agents chosen from optionally saturated, linear and branched surface-active agents comprising fatty chains having 8 to 22 carbon atoms, wherein said at least one dispersing and stabilizing agent is present in an amount ranging from 0.05% to 3% by weight relative to the total weight of said composition and further wherein said surface-active agents are water-soluble at room temperature.

40. A composition according to claim 39, wherein said mixture comprises:
(a) phytanetriol in an amount ranging from 10% to 30% by weight relative to the total weight of said mixture; and
(b) at least one unsaturated fatty acid monoglyceride in an amount ranging from 70% to 90% by weight relative to the total weight of said mixture.

41. A composition according to claim 24, wherein said at least one dispersing and stabilizing agent is chosen from
polyol alkyl ethers, polyol alkenyl ethers, polyol alkyl esters, and polyol alkenyl esters;
N-acylated amino acids and derivatives thereof;
peptides, wherein said peptides are N-acylated with at least one group chosen from alkyl groups and alkenyl groups, and salts thereof;
alkyl ethers, alkenyl ethers, alkyl ester sulfates and alkenyl ester sulfates, derivatives and salts thereof;
polyoxyethylenated alkyl fatty ethers, polyoxyethylenated alkenyl fatty ethers, polyoxyethylenated alkyl fatty esters, and polyoxyethylenated alkenyl fatty esters;
polyoxyethylenated alkyl carboxylic acids, polyoxyethylenated alkenyl carboxylic acids and salts thereof;
N-alkyl betaines, and N-alkenyl betaines; and
alkyltrimethylammonium, alkenyltrimethylammonium and salts thereof.

42. A composition according to claim 41, wherein said alkyl and alkenyl comprise from 8 to 22 carbon atoms.

43. A composition according to claim 24, wherein the binder has a pH ranging from 5 to 8.

44. A composition according to claim 43, wherein the binder has a pH ranging from 6 to 7.

45. A composition according to claim 28, wherein said cubic gel particles are further formed from at least one water-insoluble ionic amphiphilic lipid.

46. A composition according to claim 45, wherein said at least one water-insoluble ionic amphiphilic lipid is present in an amount ranging from 0.0005% to 5% by weight relative to the total weight of said composition.

47. A composition according to claim 45, wherein said at least one water-insoluble ionic amphiphilic lipid is present in an amount ranging from 0.001% to 2% by weight relative to the total weight of said composition.

48. A composition according to claim 45, wherein said at least one water-insoluble ionic amphiphilic lipid is chosen from natural, modified and synthetic phospholipids, phosphoric esters of fatty alcohols, N-acylated derivatives of glutamic acid, sodium cetyl sulphate, sodium cocoyl monoglyceride sulphate and quaternary ammonium derivatives.

49. A composition according to claim 24, wherein said aqueous continuous phase further comprises at least one active principle chosen from hydrophilic active principles and lipophilic active principles.

50. A composition according to claim 49, wherein said at least one active principle is chosen from:
antioxidants and free-radical scavengers,
moisturizing and wetting agents,
UV screening agents,
keratolytic agents,
tanning accelerators and tyrosine derivatives,
depigmenting agents,
natural colorants,
self-tanning agents,
liporegulators,
anti-ageing and anti-wrinkle agents,
anti-inflammatories and cicatrizing agents,
antibacterial and antifungal agents,
insect repellents,
deodorants,
antidandruff agents,
agents for preventing hair loss,
hair dyes,
permanent-wave reducing agents,
skin and hair conditioners, and
mixtures thereof.

51. A composition according to claim 22, wherein the lyotropic liquid crystals are lamellar liquid crystals.

52. A composition according to claim 51, wherein the binder comprises a dispersion of vesicles provided with a lamellar liquid crystal coating.

53. A composition according to claim 52, wherein the vesicles have an oily core.

54. A composition according to claim 53, wherein said oily core of said vesicles comprises at least one material chosen from oils, lipophilic active agents dissolved in oil, and active agent dissolved in oil.

55. A composition according to claim 53, wherein the dispersion of vesicles with an oily core provided with a lamellar liquid crystal coating comprises an emulsion of oil-in-water type comprising oily globules each with a lamellar liquid crystal coating and dispersed in an aqueous phase.

56. A composition according to claim 55, wherein each oily globule is individually coated with a monolamellar or oligolamellar layer obtained from at least one lipophilic surfactant, from at least one hydrophilic surfactant and from a compound chosen from i) fatty acids and ii) ionic amphiphilic lipids giving the emulsion a pH ranging from 5.5 to 7.5.

57. A composition according to claim 56, wherein the at least one lipophilic surfactant and the at least one hydrophilic surfactant each comprise at least one saturated fatty chain comprising more than about 12 carbon atoms.

58. A composition according to claim 57, wherein the fatty acid is a saturated fatty acid comprising from 16 to 22 carbon atoms.

59. A composition according to claim 56, wherein the at least one lipophilic surfactant has an HLB ranging from about 2 to about 5.

60. A composition according to claim 59, wherein said at least one lipophilic surfactant is chosen from sucrose distearate, diglyceryl distearate, tetraglyceryl tristearate, decaglyceryl decastearate, diglyceryl monostearate, hexaglyceryl tristearate, decaglyceryl pentastearate, sorbitan monostearate, sorbitan tristearate, diethylene glycol monostearate, the glyceryl ester of palmitic acid, the glyceryl ester of stearic acid, 2 EO polyoxyethylenated monostearate, glyceryl monobehenate and glyceryl dibehenate, and pentaerythrityl tetrastearate.

61. A composition according to claim 56, wherein said at least one hydrophilic surfactant has an HLB ranging from about 8 to about 12.

62. A composition according to claim 61, wherein said at least one hydrophilic surfactant is chosen from 4 EO polyoxyethylenated sorbitan monostearate, 20 EO polyoxyethylenated sorbitan tristearate, 8 EO polyoxyethylenated monostearate, hexaglyceryl monostearate, 10 EO polyoxyethylenated monostearate, 12 EO polyoxyethylenated distearate, and 20 EO polyoxyethylenated methylglucose distearate.

63. A composition according to claim 56, wherein said ionic amphiphilic lipids are chosen from neutralized anionic lipids, amphoteric lipids, and alkylsulphonic derivatives.

64. A composition according to claim 63, wherein said neutralized anionic lipids are chosen from alkali metal salts of dicetyl phosphate, alkali metal salts of dimyristyl phosphate, alkali metal salts of cholesteryl sulphate, alkali metal salts of cholesteryl phosphate, monosodium salts of acylglutamic acids, disodium salts of acylglutamic acids, and the sodium salt of phosphatidic acid.

65. A composition according to claim 63, wherein said amphoteric lipids are chosen from phospholipids.

66. A composition according to claim 63, wherein said alkylsulphonic derivatives have the formula: 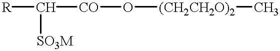
in which R is chosen from C₁₆H₃₃ groups and C₁₈H₃₇ groups and mixtures thereof and M is chosen from alkali metals.

67. A composition according to claim 55, wherein the coating of the oily globules comprises from about 2% to about 6% by weight relative to the total weight of the binder.

68. A composition according to claim 67, wherein the coating of the oily globules comprises from 3% to 4% by weight relative to the total weight of the binder.

69. A composition according to claim 56, wherein the relative amounts of at least one lipophilic surfactant, at least one hydrophilic surfactant and said compound chosen from fatty acids and ionic amphiphilic lipids range respectively from: 35-55%,25-40%/15-35% by weight relative to their total weight.

70. A composition according to claim 55, wherein the coated oily globules are present in an amount ranging from 5% to 50% by weight relative to the total weight of the binder.

71. A composition according to claim 70, wherein the coated oily globules are present in an amount ranging from 10% to 40% by weight relative to the total weight of the binder.

72. A composition according to claim 55, wherein the ratio of the weight of the oily globules to the weight of the coating ranges from 2:1 to 13:1.

73. A composition according to claim 71, wherein the ratio of the weight of the oily globules to the weight of the coating is about 7:1.

74. A composition according to claim 55 further comprising a basic agent.

75. A composition according to claim 74 wherein the basic agent is chosen from sodium hydroxide, triethanolamine, lysine, arginine, and mixtures thereof.

76. A composition according to claim 52, wherein the vesicles comprise an aqueous core.

77. A composition according to claim 76, wherein the vesicles comprising an aqueous core provided with a lamellar liquid crystal coating are obtained from at least one amphiphilic lipid having the general formula:
X-Y
in which X represents a hydrophilic group and Y represents a lipophilic group.

78. A composition according to claim 77, wherein the vesicles comprise a lipid-phase membrane encapsulating an aqueous phase, the lipid phase comprising at least one nonionic amphiphilic lipid and at least one ionic amphiphilic lipid.

79. A composition according to claim 78, wherein:
the at least one nonionic amphiphilic lipid is chosen from esters formed from (a) at least one polyol chosen polyethylene glycol bearing from 1 to 60 ethylene oxide units, sorbitan, sorbitan bearing 2 to 60 ethylene oxide units, glycerol bearing 2 to 30 ethylene oxide units, polyglycerols comprising 2 to 15 glycerol units, sucroses, glucoses bearing 2 to 30 ethylene oxide units, and (b) at least one fatty acid comprising an alkyl chain chosen from linear and branched, saturated and unsaturated C5-C17 alkyl chains, the number of alkyl chains per polyol group ranging from 1 to 10; and
the at least one ionic amphiphilic lipid is chosen from ionic amphiphilic lipids giving the dispersion a pH ranging from 5.5 to 7.5;
the weight ratio of the at least one nonionic amphiphilic lipid to the at least one ionic amphiphilic lipid in the lipid phase ranging from 50:1 to 50:25 and the weight ratio of the lipid phase to the aqueous phase of the dispersion ranging from 1:1,000 to 300:1,000.

80. A composition according to claim 78, wherein the at least one ionic amphiphilic lipid combined with the nonionic amphiphilic lipids is chosen from:
alkali metal salts of dicetyl phosphate and alkali metal salts of dimyristyl phosphate;
alkali metal salts of cholesteryl sulphate;
alkali metal salts of cholesteryl phosphate;
lipoamino acids and salts thereof;
the sodium salts of phosphatidic acid;
phospholipids; and
alkylsulphonic derivatives of the formula: 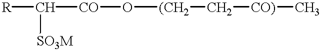
in which R is chosen from C₁₆-C₂₂ alkyl groups, and M is chosen from alkali metals and alkaline-earth metals.

81. A composition according to claim 80 wherein R is chosen from C₁₆H₃₃ groups and C₁₈H₃₇ groups, and mixtures thereof.

82. A composition according to claim 78, wherein the lipid phase comprises at least one additive chosen from sterols, C₁₄ to C₃₀ alcohols, C₁₄ to C₃₀ diols, C₁₄ to C₃₀ amines and quaternary ammonium derivatives thereof.

83. A composition according to claim 77 wherein the vesicles have a mean diameter ranging from 10 to 5,000 nm.

84. A composition according to claim 77 wherein the vesicles contain at least one active compound.

85. A composition according to claim 1, wherein the binder comprises an O/W emulsion comprising at least one surfactant with a surface tension of greater than or equal to 50 mN/m.

86. A composition according to claim 85, wherein the at least one surfactant is chosen from stearic acid combined with triethanolamine, the mixture of glyceryl monostearate and glyceryl distearate, sodium lauroyl sarcosinate, cetearyl glucoside, PEG-40 stearate, sorbitan tristearate, sorbitan stearate, polysorbate-60, the mixture sorbitan stearate/sucrose cocoate, the mixture glyceryl stearate/PEG-100 stearate, PEG-400, glyceryl stearate, and the mixture PEG-6/PEG-32/glycol stearate.

87. A composition according to claim 1, wherein the binder comprises at least one oil chosen from mink oil, turtle oil, soybean oil, grape pip oil, sesame oil, corn oil, rapeseed oil, sunflower oil, cotton oil, avocado oil, olive oil, castor oil, jojoba oil, groundnut oil, hydrocarbon oils, fatty esters, perfluoro oils, higher fatty acids, higher fatty alcohols, and silicone oils.

88. A composition according to claim 1, wherein the binder comprises at least one fatty substance chosen from waxes and pasty fatty substances.

89. A composition according to claim 88, wherein the pasty fatty substance has a viscosity of from 0.1 to 40 Pa.s (1 to 400 poises), measured at 40° C. with a Contraves TV rotary viscometer equipped with an MS-r3 or MS-r4 spindle, at a frequency of 240 rpm.

90. A composition according to claim 89, wherein the pasty fatty substance has a viscosity of 0.5 to 25 Pa.s.

91. A composition according to claim 88, wherein the pasty fatty substance has a melting point of 25-70° C.

92. A composition according to claim 91, wherein the pasty fatty substance has a melting point of 25-55° C.

93. A composition according to claim 88, wherein said waxes are chosen from beeswaxes, lanolin waxes and Chinese insect waxes; carnauba wax, candelilla wax, ouricurry wax, cork fibre waxes, sugar cane waxes, Japan waxes, hydrogenated jojoba waxes, paraffins, microcrystalline waxes, montan waxes, ozokerites; polyethylene waxes, the waxes obtained by Fischer-Tropsch synthesis, waxy copolymers and esters thereof, and silicone waxes.

94. A composition according to claim 1, wherein the binder further comprises a volatile compound chosen from hydrocarbon-based oils, cyclic volatile silicones comprising from 3 to 8 silicon atoms, and linear volatile silicones comprising from 2 to 9 silicon atoms.

95. An eyeshadow, blusher, face powder, body powder, concealer product or make-up product for the body comprising a powder comprising a pulverulent phase and a binder, said binder being chosen from compositions comprising an aqueous continuous phase.

96. A process for making up or caring for keratinous materials of human beings, comprising applying to said keratinous materials a composition in the form of a powder comprising a pulverulent phase and a binder in the powder composition, wherein the binder is chosen from compositions comprising an aqueous continuous phase.

97. A process for preparing a composition in the form of a powder comprising a pulverulent phase and a binder in the powder composition, wherein the binder is chosen from compositions comprising an aqueous continuous phase, said process comprising the following steps:
a) mixing together at least one pulverulent compound,
b) grinding the mixture obtained in a) into fine particles, and
c) adding said binder and mixing.

98. A composition according to claim 1, wherein said aqueous continuous phase comprises cubic gel particles formed from at least:
(i) phytanetriol, wherein said phytanetriol is present in an amount ranging from 0.1% to 15% by weight relative to the total weight of the binder, and
(ii) at least one dispersing and stabilizing agent chosen from surface-active agents chosen from optionally saturated, linear and branched surface active agents comprising fatty chains having from 8 to 22 carbon atoms, wherein said at least one dispersing and stabilizing agent is present in an amount ranging from 0.1% to 3% by weight relative to the total weight of said binder and further wherein said surface-active agents are water-soluble at room temperature.

99. A composition according to claim 50, wherein
said antioxidants and free-radical scavengers are chosen from proteins, enzymes, lactoperoxidase, lactoferrin, peptides and derivatives thereof, sequestrants, flavonoids, chlorophylline, ethoxyquine, guanosine, tocopherols and derivatives thereof, ascorbyl palmitate, β-carotene, vitamin E and derivatives thereof, vitamin C and derivatives thereof and vitamin A and derivatives thereof,
said moisturizing and wetting agents are chosen from hyaluronic acid, and its sodium salt, β-glycerophosphate, glycerol, sorbitol and panthenol,
said UV screening agents are chosen from the products sold under the names “Eusolex 232®” by the company Merck, “Parsol 1789®” and “Parsol MCX®” by the company Givaudan-Rouré, “Mexoryl SX®” by the company Chimex and “Uvinul T1500®” by the company BASF,
said keratolytic agents are chosen from proteolytic enzymes, salicylic acid and derivatives thereof, and retinoic acid and derivatives thereof,
said tanning accelerators are chosen from caffeine, and tyrosine derivatives,
said depigmenting agents are chosen from kojic acid, glycolic acid, vitamin C, magnesium ascorbyl phosphate, arbutin, and derivatives thereof,
said natural colorants are chosen from dyestuffs extracted from plants, and dyestuffs extracted from animals,
said self-tanning agents are chosen from dihydroxyacetone and indoles,
said liporegulators are chosen from γ-orizanol, extract of Centella asiatica comprising genin and asiatic acid, caffeine and theophylline,
said anti-ageing and anti-wrinkle agents are chosen from hydroxy acids,
said antiinflammatories and cicatrizing agents are chosen from α-bisabolol, corticoids, 18-β-glycyrrhetinic acid and its salts extract of Centella asiatica, and aloe vera,
said antibacterial and antifungal agents are chosen from benzalkonium chloride, chlorhexidine, hexetidine and hexamidine,
said insect repellents are chosen from diethyltoluamides and dimethyltoluamides,
said deodorants are chosen from hexachlorophene, and the product Triclosan sold under the name “Irgasan DP 300®” by the company Ciba Geigy,
said antidandruff agents are chosen from octopirox, and pyridinethione derivatives,
said agents for preventing hair loss are chosen from methyinicotinate, hexylnicotinate, and Minoxidil,
said hair dyes are chosen from oxidation bases, oxidation couplers, direct dyes and self-oxidizing dyes,
said permanent-wave reducing agents are chosen from thioglycolic acid, cysteine, cysteamine, n-acetylcysteine, N-acetylcysteamine and glyceryl thioglycolate, and
said skin and hair conditioners chosen from cationic polymers and cations.

100. A composition according to claim 80, wherein
said alkali metal salts of dicetyl phosphate are chosen from the sodium and potassium salts;
said alkali metal salts of dimyristyl phosphate are chosen from the the sodium and potassium salts;
said alkali metal salts of cholesteryl sulphate are chosen from the sodium salt;
said alkali metal salts of cholesteryl phosphate are chosen from the sodium salt;
said monosodium salts of acylglutamic acids and disodium salts of acylglutamic acids are chosen from the monosodium salts of N-stearoylglutamic acid and the disodium salts of N-stearoylglutamic acid, and
in said alkylsulphonic derivatives of the formula: 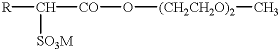
M is chosen from sodium.

101. A composition according to claim 1, wherein the pulverulent phase comprises flakes.

102. A process for improving the moisturization of a make-up and cosmetic care composition in powder form comprising:
incorporating into said composition at least one aqueous continuous phase in an amount sufficient to improve said moisturiztion.

103. A process for improving the development of color in a make-up and cosmetic care composition in powder form comprising:
incorporating into said composition at least one aqueous continuous phase in an amount sufficient to improve said color development.
